(19) United States
(12) Patent Application Publication
(10) Pub. No.: US 2017/0325977 A1
(43) Pub. Date: Nov. 16, 2017

(54) Endovascular Graft Defining Internal Lumens

(71) Applicant: Yale University, New Haven, CT (US)

(72) Inventors: Timur Sarac, Milford, CT (US); Jason Chin, New Haven, CT (US)

(73) Assignee: Yale University, New Haven, CT (US)

(21) Appl. No.: 15/596,250

(22) Filed: May 16, 2017

Related U.S. Application Data
(60) Provisional application No. 62/337,039, filed on May 16, 2016.

(51) Int. Cl. A61F 2/856; A61F 2/90; A61F 2/92

(52) U.S. Cl. CPC A61F 2/856; A61F 2/92; A61F 2/90; A61F 2220/0008

(57) ABSTRACT

An endovascular graft is provided that includes a stent structure adapted to move between a collapsed and a deployed configuration. An endovascular graft material is mounted with respect to the stent structure. At least one lumen- or chute-forming structure is associated with the stent structure internal to the endovascular graft material. The at least one lumen- or chute-forming structure is adapted to move between a first position and a second position, wherein the at least one lumen- or chute-forming structure is rolled within itself in the first position, and wherein the at least one lumen- or chute-forming structure moves from the first position to the second position by unrolling. When in the second position, the at least one lumen- or chute-forming structure defines a passage having a greater cross-sectional area as compared to the first position. Stent-graft deployment to one or more branch arteries/vessels may be accomplished through the at least one lumen- or chute-forming structure. The present disclosure also provides advantageous methods for deploying the disclosed endovascular graft and delivery of stent-grafts to branch arteries/vessels therethrough.

### CROSS-REFERENCE TO RELATED APPLICATION

The present application claims priority benefit to a provisional patent application entitled “Endovascular Graft Defining Internal Lumens,” which was filed on May 16, 2016, and assigned Ser. No. 62/337,039. The entire content of the foregoing provisional patent application is incorporated herein by reference.

### BACKGROUND

### 1. Technical Field

The present disclosure is directed to endovascular graft designs, systems and methods that include or define, inter alia, one or more lumens that facilitate treatment of one or more branch arteries. The disclosed endovascular graft designs, systems and methods advantageously obviate potential leaks associated with conventional chimney techniques.

### 2. Background Art

An aneurysm generally involves an expansion and weakening of the wall of an artery. Thoracic aortic aneurysms, thoraco-abdominal aortic aneurysms, or abdominal aortic aneurysms present serious and potentially life threatening conditions. More specifically, juxtarenal abdominal aortic aneurysms (JAAAs) and thoracobdominal aortic aneurysms (TAAAs) present challenging surgical problems whose solutions thus far have been fraught with high rates of morbidity and mortality compared to infrarenal abdominal aortic aneurysms. Direct repair through open surgery, while held to be the long-term gold standard solution, is far from a routine surgical endeavor. Even when successful, direct open surgical repair procedures generally require significant patient recovery time, intensive care, and expenditures.

In contrast, infrarenal endovascular aortic repair (EVAR) can be performed entirely through endovascular means, and even percutaneously with minimal recovery for the patient and significantly reduced requirements for perioperative care. However, JAAAs and TAAAs repair by conventional EVAR grafts raise significant issues due to the need for branch artery preservation. For example, preservation of the left subclavian vessel when undertaking treatment of a thoracic aneurysm, renal branch vessels when treating abdominal aneurysms, and superior mesenteric and celiac arteries when treating TAAAs is important.

Currently, a chimney (or sometimes referred to as “periscope”) technique is utilized in an effort to achieve branch artery preservation, whereby additional stents are placed simultaneously adjacent to the EVAR graft and into otherwise covered branch vessels to maintain perfusion.

Separate chimney stent-grafts are generally positioned between the aortic wall and main endovascular graft, and the chimney stent-grafts are generally positioned in the body before the main graft is deployed, so if difficulty is encountered in chimney deployment, the chimney stent-grafts may be repositioned and/or redeployed before deployment of the main endovascular graft. The chimney technique is unfortunately vulnerable to significant risk of leakage through “gutters” between adjacent grafts, and further enlargement of the aneurysm. Indeed, the chimney technique typically gives rise to regions of blood leakage because the main endovascular graft cannot conform fully to the geometry of the chimney stent-grafts which must be stiff enough to resist collapse and prevent branch vessel occlusion. Such blood leakage, if it occurs, can result in clinical problems and/or failure of the endovascular graft.

Fenestrated EVAR grafts with separate stent-grafts or stents deployed within the branch vessel and through the graft fenestrations have been proposed to address the need for branch artery preservation and as a possible solution to gutter leakage. However, fenestrated EVAR grafts are limited by their need for individualized-design which is an expensive, time-consuming process and which cannot be achieved in urgent situations.

Commercially available endoprostheses for the endovascular treatment of abdominal aortic aneurysms include the Endurant® stent-graft system (Medtronic, Inc.; Minneapolis, Minn.), the Zenith® stent-graft system (Cook, Inc.; Bloomington, Ind.), the PowerLink® stent-graft system (Endologix, Inc.; Irvine, Calif.), and the Excluder® stent-graft system (W.L. Gore & Associates, Inc.; Newark, Del.) amongst others. A commercially available stent-graft for the treatment of thoracic aortic aneurysms is the TAG™ system (W.L. Gore & Associates, Inc.; Newark, Del.), TX2 system (Cook, Inc.; Bloomington, Ind.), and VALOR system (Medtronic, Inc.; Minneapolis, Minn.).

Given the above-noted constraints, JAAAs and TAAAs are generally repaired via open surgery. Thus, a need remains for devices, systems and methods that permit effective endovascular repair, preserve branch artery patency, and avoid (or substantially reduce) leakage relative to the endovascular graft deployment. These and other objects are satisfied by the endovascular graft devices, systems and methods of the present disclosure.

### SUMMARY OF THE INVENTION

The present invention relates generally to devices, systems and methods for the treatment of disorders of the vasculature, particularly aneurysms. More particularly, the present disclosure provides advantageous devices, systems and methods for treating diseased bodily lumens involving branched lumen deployment sites. The disclosed devices/systems generally include a main endovascular graft that includes a collapsible stent structure to which is mounted/attached an endovascular graft material, thereby defining inner and outer endovascular graft regions. The main endovascular graft is configured and dimensioned for deployment in a main artery, e.g., in connection with JAAA and/or TAAA repair. The disclosed devices/systems further include one or more internally positioned lumen- or chute-forming structures that are joined/attached with respect to the stent structure and that are adapted for deployment to define substantially cylindrical passage(s) positioned internal to the main endovascular graft. When deployed, the lumen- or chute-forming structure(s) define passages for introduction of stent-graft(s) for deployment in branch vessels/arteries therethrough.

According to exemplary embodiments of the present disclosure, each of the lumen- or chute-forming structures defines an interrupted cylinder and is adapted to be “rolled up” so as to reduce the volume/space occupied thereby, e.g., during deployment of the disclosed device/system in a bodily lumen such as the aorta. The are fixedly joined relative to the stent structure. Thus, the lumen- or chute-forming structures may be welded with respect to the stent structure, e.g., along a first edge of the cylindrical structure, or formed by laser-cutting technology. Thus, the opposite/free edge of the cylindrical structure may roll up within itself to define a cylinder of reduced cross-section, and may thereafter “unroll” to define a cylinder of increased cross-section.

The lumen- or chute-forming structures are fixedly joined relative to the stent structure so as to ensure a secure connection therebetween.

The disclosed device/system generally defines a longitudinal axis that aligns with the main vessel to be treated according to the present disclosure. The first edge (i.e., the edge that is fixedly joined relative to the stent structure) of each of the interrupted cylinders that define the lumen- or chute-forming structures is generally aligned with such longitudinal axis. When rolled up relative to itself, the lumen- or chute-forming structure defines a spiral-shaped cross-section that opens at proximal and distal ends thereof. The stent structure to which the lumen- or chute-forming structure(s) are joined also collapses relative to itself, e.g., defining a substantially star-shaped cross-section.

When deployed in a vessel, the disclosed device/system “opens” or expands such that the outer graft material engages the vessel wall. Within the device/system, the stent structure transitions from a collapsed “star-shaped cross-section” to an expanded cross-section which approaches a substantially circular cross-section. Within the substantially circular cross-section, one or more discrete lumens are defined by the lumen- or chute-forming structures that are joined to the stent-structure along the longitudinal axis thereof. In exemplary embodiments of the present disclosure, two to four lumen- or chute-forming structures are joined to the stent structure to accommodate the introduction/deployment of stent-grafts in branch vessels/arteries, although the present disclosure is not limited by or to the noted exemplary implementations. Indeed, as few as one lumen- or chute-forming structure may be associated with the disclosed stent structure, and more than four lumen- or chute-forming structures may be provided according to the present invention. The plurality of lumen- or chute-forming structures are spaced around the cross-section of stent structure of the device/system, e.g., based on typical anatomical spacing of branch vessels/arteries.

When “rolled up,” the lumen- or chute-forming structures are substantially biased to deploy into a non-rolled up configuration, i.e., to define lumens with substantially circular cross-sections and limited spiraling of the interrupted cylindrical structure. To the extent the noted bias is insufficient to fully deploy/unroll the lumen- or chute-forming structure(s), further deployment/unrolling may be effectuated when the surgeon introduces a stent-graft to one end thereof. The lumen- or chute-forming structures advantageously define discrete passages for safe and efficient introduction of stent-grafts that may pass through the main stent graft for introduction to desired branch vessels/arteries. Because these discrete passages are part of the wall of the main stent graft, there is no risk of leakage around or relative to these lumen- or chute-forming structures; rather, the integrity of engagement between the endovascular graft material and the inner wall of the main vessel is maintained/not interrupted by the introduction of one or more stent-grafts to branch vessels/arteries through the internal lumens defined by the lumen- or chute-forming structures.

When deployed, the disclosed device/system provides a main stent graft that permits blood flow through the lumen/interior region defined by the endovascular graft material which is engaged with the inner wall of the main vessel. The available flow path of the lumen/interior region of the main stent graft is generally reduced by the cross-sectional area of the discrete lumens defined by the lumen- or chute-forming structures. The interrupted cylindrical geometries of the discrete lumen- or chute-forming structures open radially outward. Blood flow from the main artery to the branch artery (or branch arteries) is facilitated by the lumen- or chute-forming structures and the associated stent-grafts positioned within the branch artery/arteries.

Thus, the present invention provides endovascular graft devices/systems and associated methods for aneurysm treatments that include or involve branch vessels. Additional features, functions and benefits of the disclosed devices, systems and methods will be apparent from the detailed description which follows, particularly when read in conjunction with the appended figures.

### BRIEF DESCRIPTION OF THE FIGURES

To assist those of skill in the art in making and using the disclosed devices, systems and methods, reference is made to the accompanying figures, wherein:

FIG. 1 is a schematic illustration of an abdominal aortic repair endovascular graft system associated with a stent structure according to the present disclosure.

FIGS. 2 and 3 are perspective schematic views of exemplary endovascular graft devices/systems for treating diseased vasculature, e.g., aneurysms, according to the present disclosure;

FIGS. 4A, 4B and 4E are schematic side views of an exemplary endovascular graft device/system for treating diseased vasculature according to the present disclosure;

FIGS. 4C and 4D are schematic top/bottom views of the exemplary endovascular graft device/system of FIG. 4B in a deployed configuration;

FIG. 5 is a schematic top view of an exemplary endovascular graft device/system in a non-deployed configuration according to the present disclosure;

FIGS. 6A, 6B and 6C are schematic end views of an exemplary embodiment of the disclosed endovascular graft device/system at various points of deployment;

FIGS. 7A, 7B, 7C and 7D are further schematic end views of an exemplary embodiment of the disclosed endovascular graft device/system at various points of deployment;

FIG. 8 is a schematic view of an exemplary endovascular graft in a deployed orientation according to the present disclosure; and

FIGS. 9A, 9B, 9C and 9D are schematic perspective views of an exemplary embodiment of the disclosed endovascular graft device/system in a fully expanded/deployed configuration; in FIG. 9A, the outer graft material is removed for illustration purposes; in FIG. 9B, the outer graft material is included in the schematic depiction; in FIG. 9C, the exemplary endovascular graft is shown in an exemplary anatomical orientation/position; and in FIG. 9D, a stent-graft is introduced to a branch vessel/artery through one of lumen- or chute-forming structures.

### DESCRIPTION OF EXEMPLARY EMBODIMENT(S)

As noted above, the present invention provides advantageous devices, systems and methods for treating diseased bodily lumens and for the treatment of disorders of the vasculature, particularly aneurysms, e.g., vasculature that includes branched vessels and/or arteries requiring stent-graft deployment.

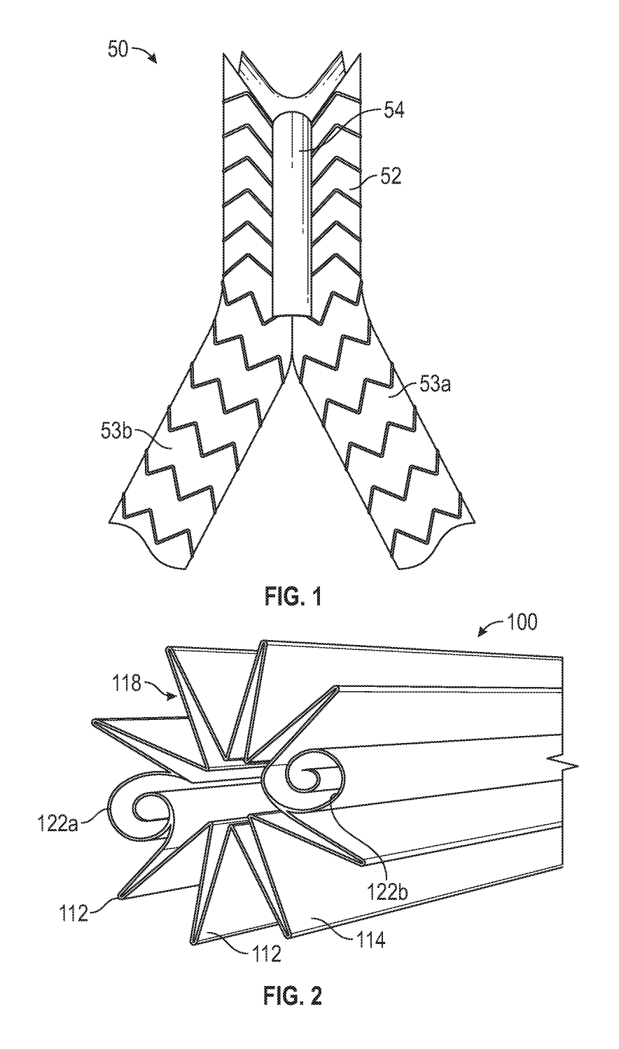

With reference to FIG. 1, an exemplary endovascular graft system 50 that includes a main endovascular graft segment 52 and first/second branches 54a, 54b is schematically depicted. The disclosed endovascular graft system 50 may be used in repair of a thoracic abdominal aneurysm or an abdominal aortic aneurysm, e.g., JAAA or TAAA procedures. Of note, the disclosed endovascular graft system 50 includes and is supported by a stent structure 52 that defines one or more lumen- or chute-forming structures, as further described below.

With reference to FIGS. 2 and 3, an exemplary endovascular graft 100 is provided that includes a collapsible stent structure 112 to which is mounted/attached an endovascular graft material 114, thereby defining inner endovascular graft region 116 and an outer region 118. The endovascular graft 100 is configured and dimensioned for deployment in a main artery, e.g., in connection with JAAA and/or TAAA repair.

Endovascular graft 100 further includes one or more internally positioned lumen- or chute-forming structures 120 that are joined/attached with respect to the stent structure 112 and that are adapted for deployment to define substantially cylindrical passage(s) 122a, 122b positioned internal to the main endovascular graft 100. When deployed, the lumen- or chute-forming structure(s) 120 define passages for introduction of stent-graft(s) for deployment in branch vessels/arteries therethrough.

According to exemplary embodiments of the present disclosure, the lumen- or chute-forming structures 120 define substantially cylindrical structures that are adapted to be “rolled up” (as shown in FIGS. 2 and 3) so as to reduce the volume/space occupied thereby, e.g., during deployment of the disclosed device/system 100 in a bodily lumen such as the aorta. When rolled up, exemplary lumen- or chute-forming structures have a reduced cross-sectional diameter, e.g., on the order of 3 mm, and when fully deployed, the lumen- or chute-forming structures define an increased cross-sectional diameter, e.g., on the order of 8 mm to 10 mm. Thus, in exemplary implementations, the overall diameter of the lumen- or chute-forming structures may be reduced by a factor of 2 to 5 (or even more), although the present disclosure is not limited by or to such exemplary dimensional properties, but may be varied to address clinical objectives, as will be readily apparent to persons skilled in the art. Indeed, variously sized lumen- or chute-forming structures may be associated with a single endovascular graft, thereby optimizing lumen dimensions to accommodate stent-graft deployment in branch vessels of varying size and geometry.

The lumen- or chute-forming structures 120 may be laser cut or welded with respect to the stent structure 112, e.g., along a first edge of the interrupted cylindrical structure of the lumen- or chute-forming structure, with the opposite edge freely suspended. Thus, the opposite/free edge of the interrupted cylindrical structure may roll up within itself to define a cylinder of reduced cross-section, as generally shown in FIGS. 2 and 3. Fabrication of the disclosed endovascular graft using a laser cutting process is generally effective to provide structural integrity between the lumen- or chute-forming structures and the stent structure of the present disclosure.

The endovascular graft 100 generally defines a longitudinal axis that, when used clinically, aligns with the main vessel to be treated according to the present disclosure. The axes of the lumen- or chute-forming structures are generally aligned with such longitudinal axis. When rolled up relative to itself, each lumen- or chute-forming structure defines a substantially spiral-shaped cross-section that is open at proximal and distal ends thereof.

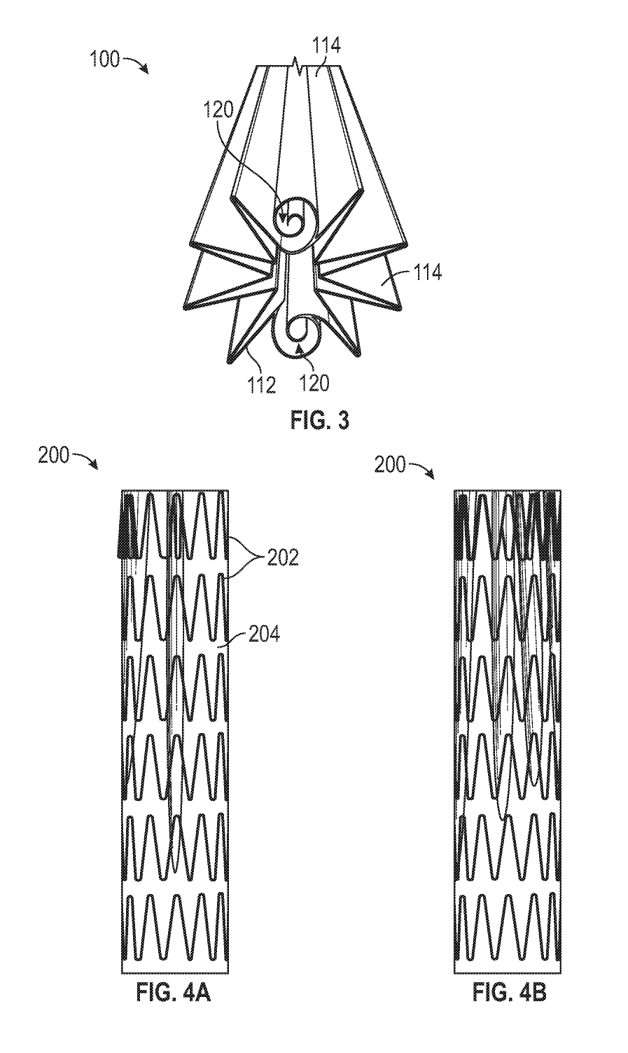

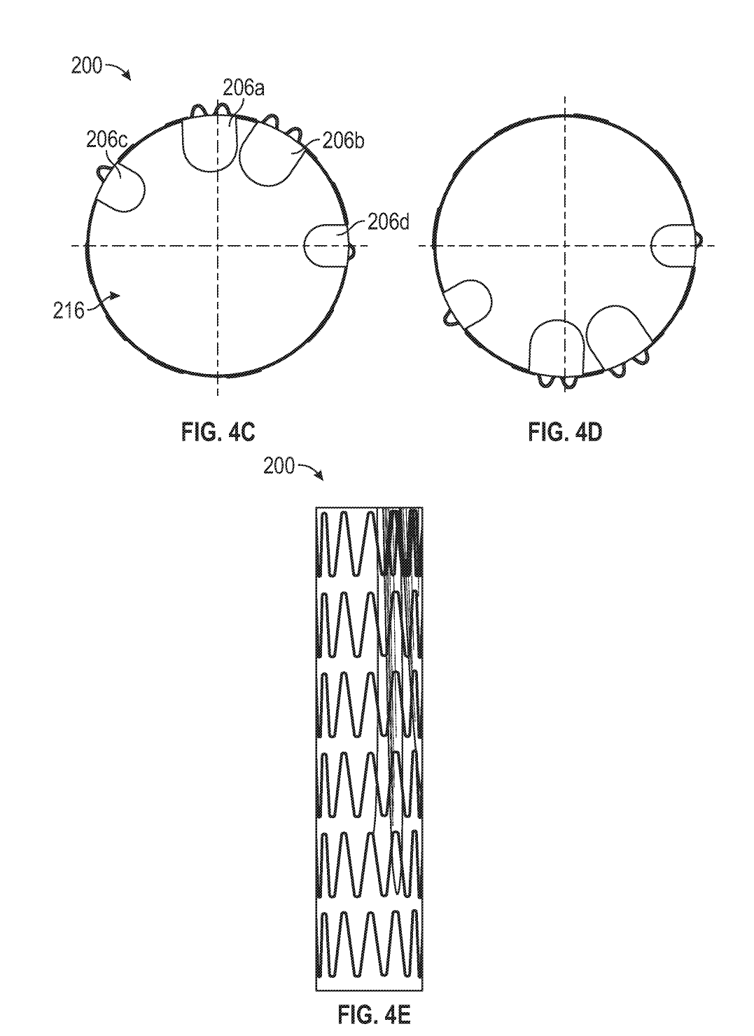

With reference to FIGS. 4A-4E, a series of schematic views of a further exemplary endovascular graft 200 according to the present disclosure are provided. Endovascular graft 200 includes a stent structure 202 that supports endovascular graft material 204. With particular reference to FIGS. 4C and 4D, the top/bottom views of the endovascular graft 200 as shown in FIG. 4B depict four (4) distinct lumen- or chute-forming structures 206a-206d in their substantially “deployed” or “expanded” condition. Thus, exemplary radial spacing of lumen- or chute-forming structures 206a-206d is schematically depicted, with larger lumen- or chute-forming structures 206a, 206b surrounded by relatively smaller lumen- or chute-forming structures 206c, 206d. More particularly, within inner region 216 of endovascular graft 200 (which defines a substantially circular cross-section), four discrete lumens are defined by the lumen- or chute-forming structures 206a-206d that are joined to or integrally formed with the stent-structure 202 along the longitudinal axis thereof.

As noted above, in exemplary embodiments of the present disclosure, two to four lumen- or chute-forming structures are associated with the endovascular graft to accommodate the introduction/deployment of stent-grafts in branch vessels/arteries, although fewer or greater numbers of lumen- or chute-forming structures may be incorporated into the disclosed endovascular graft of the present disclosure. Thus, in exemplary implementations of the present disclosure, from one to six lumen- or chute-forming structures may be advantageously provided.

When deployed/expanded in the manner depicted in FIGS. 4A-4E, the disclosed endovascular graft 200 accommodates stent-graft deployment through lumen- or chute-forming structures 206a-206d to branch vessels.

When deployed in a vessel, the endovascular graft 200 “opens” or expands as schematically depicted in FIGS. 4A such that the outer graft material 204 engages a vessel wall (not shown).

In undertaking such deployment, the stent structure 202 transitions from a collapsed “star-shaped cross-section” to an expanded cross-section which approaches a substantially circular cross-section.

The plurality of lumen- or chute-forming structures are radially spaced around the stent structure of the endovascular graft, e.g., based on typical anatomical spacing of branch vessels/arteries.

For example, in implementations that include two lumen- or chute-forming structures, the spacing may be about 1° to 359°. In implementations that include three lumen- or chute-forming structures, the spacing may be radially equidistant or radially non-equidistant. Typical radial spacings for implementations that include three lumen- or chute-forming structures range from 1° to 359°. In implementations that include four lumen- or chute-forming structures, the spacing may be radially equidistant or radially non-equidistant. Typical radial spacings for implementations that include four lumen- or chute-forming structures range from 1° to 359°.

When “rolled up,” the lumen- or chute-forming structures are generally biased to deploy into a non-rolled up configuration, i.e., to define lumens with substantially circular cross-sections and limited post-deployment spiraling of the rectangular structure, as schematically depicted in FIGS. 4C and 4D. To the extent the noted bias is insufficient to fully deploy/unroll the lumen- or chute-forming structure(s), further deployment/unrolling may be effectuated when the surgeon introduces a stent-graft to one end thereof, e.g., into a passage by a camming action. The lumen- or chute-forming structures advantageously define discrete passages for safe and efficient introduction of stent-grafts that may pass through the main stent graft for introduction to desired branch vessels/arteries (not shown). Because these discrete passages are internal to the main stent graft, there is no risk of leakage around or relative to these lumen- or chute-forming structures; rather, the integrity of engagement between the endovascular graft material and the inner wall of the main vessel is maintained/not interrupted by introduction of one or more stent-grafts to branch vessels/arteries through the internal passages defined by the lumen- or chute-forming structures.

When deployed, the disclosed endovascular graft provides a main stent graft that permits blood flow through the lumen/interior region defined by the endovascular graft material which is engaged with the inner wall of the main vessel. The available flow path of the lumen/interior region of the main stent graft is effectively reduced by the cross-sectional area of the discrete lumens defined by the lumen- or chute-forming structures. Blood flow from the main artery to the branch artery (or branch arteries) is facilitated by the lumen- or chute-forming structures and the associated stent-grafts positioned within the branch artery/arteries. Thus, the present invention provides endovascular graft devices/systems and associated methods for aneurysm treatments that include or involve branch vessels.

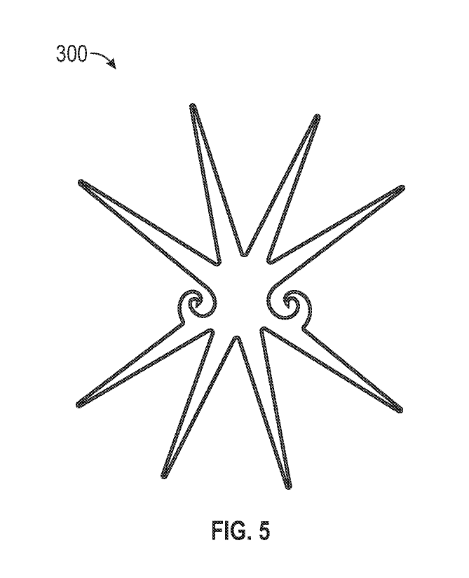

An exemplary collapsed, star-shaped configuration of an exemplary endovascular graft 300 (with two lumen- or chute-forming structures) is schematically depicted in FIG. 5. The transition from an undeployed/collapsed configuration to a deployed/expanded configuration is readily apparent from a comparison of the schematic illustrations provided herewith.

In use, the present disclosure provides an advantageous method for treating aneurysm(s) and/or providing blood flow at branched arteries that includes the steps of: (i) providing an endovascular graft (e.g., endovascular graft) in a collapsed/non-deployed configuration; (ii) positioning the endovascular graft within a main artery; and (iii) deploying the endovascular graft within the main artery, such that the endovascular graft assumes an expanded/deployed configuration and wherein one or more lumens/passages are defined within the endovascular graft to allow stent-graft deployment to one or more branch arteries/vessels. The disclosed method may further include deployment of one or more stent-grafts in one or more branch arteries/vessels thru the noted lumens/passages defined within the endovascular graft. The advantageous method for treating aneurysm(s) and/or providing blood flow at branched arteries described herein may be implemented based, in whole or in part, on the structural and functional features of the endovascular graft (e.g., endovascular graft) of the present disclosure.

The disclosed devices, systems and methods may be advantageously utilized to treat vasculature in a wide range of clinical settings, e.g., treatment of aneurysms, such as, but not limited to, thoracic aortic aneurysms and abdominal aortic aneurysms.

The endovascular graft may be formed from any known graft material that is biocompatible and durable, e.g., polytetrafluoroethylene (PTFE), polyethylene terephthalate (PET), for example DACRON (polyester), latex, balloons or sealing polymers, or biologics such as pericardium, pleura, or peritoneum, and the like. Materials of construction for the stent structure and the lumen- or chute-forming structure(s) may also be selected from known materials, e.g., stainless steel, titanium, aluminum and alloys, e.g., nitinol (NiTi) alloy. In exemplary embodiments, the stent structure and the lumen- or chute-forming structure(s) are self-expanding and/or self-deploying (in whole or in part). In forming the noted stent structure, it is contemplated that a continuous element may be used to form a series of helical windings extending from end-to-end, as is known in the art. Alternative modes of construction of the disclosed stent structure may be employed without departing from the spirit or scope of the present disclosure, as will be readily apparent to those of ordinary skill in the art.

Similarly, the endovascular graft material may be secured with respect to the stent structure and, as necessary, the lumen- or chute-forming structure(s) by conventional means, e.g., adhesive bonding. Single or multiple graft layers may be employed to ensure desired integrity of the disclosed endovascular graft. In exemplary embodiments, the endovascular graft materials has an overall thickness of about 0.003 inch to about 0.015 inch, although the present disclosure is not limited by or to such exemplary thickness range.

While the present disclosure has been described with reference to exemplary embodiments and implementations thereof, the present disclosure is not limited by or to such exemplary embodiments/implementations. Indeed, it will be appreciated that modifications and variations of the present invention may be effected by those skilled in the art without departing from the spirit and intended scope of the invention. For example, in exemplary implementations of the present disclosure, the following modifications and refinements are specifically contemplated and included within the scope of the present disclosure:
The diameters of the lumens/passages defined by the lumen- or chute-forming structure(s) when in the deployed position (e.g., the configurations of FIGS. 4C/4D) may be substantially equal or purposefully different. Thus, it is contemplated that lumens/passages of differing diameters may be incorporated into the disclosed endovascular graft in view of the different anatomical properties of associated branch vessels/arteries.
The lumen- or chute-forming structure(s) may include one or more releasable latching mechanisms that assume a latched configuration prior to deployment (e.g., the configuration of FIG. 5) and assume an unlatched configuration in order to deploy.
The disclosed endovascular graft may include indicia that aligns with the radial position of the lumen- or chute-forming structure(s) to facilitate clinical positioning, e.g., so as to radially align the lumen- or chute-forming structure(s) with the relative position of the branch vessels/arteries. The indicia may be visually discernible in various conditions and under various wavelengths of illumination.
The method of implementation may include selection of an endovascular graft based on the number of branch vessels/arteries from among an inventory of endovascular grafts fabricated according to the present disclosure. For example, if a particular surgical procedure requires the placement of stent grafts in three branch vessels/arteries, an endovascular graft that provides three lumen- or chute-forming structure(s) may be selected for use in such procedure. A variety of endovascular grafts (e.g., devices that include two internal lumens, three internal lumens and four internal lumens) may be provided in a kit to accommodate inventory control and surgical selection.

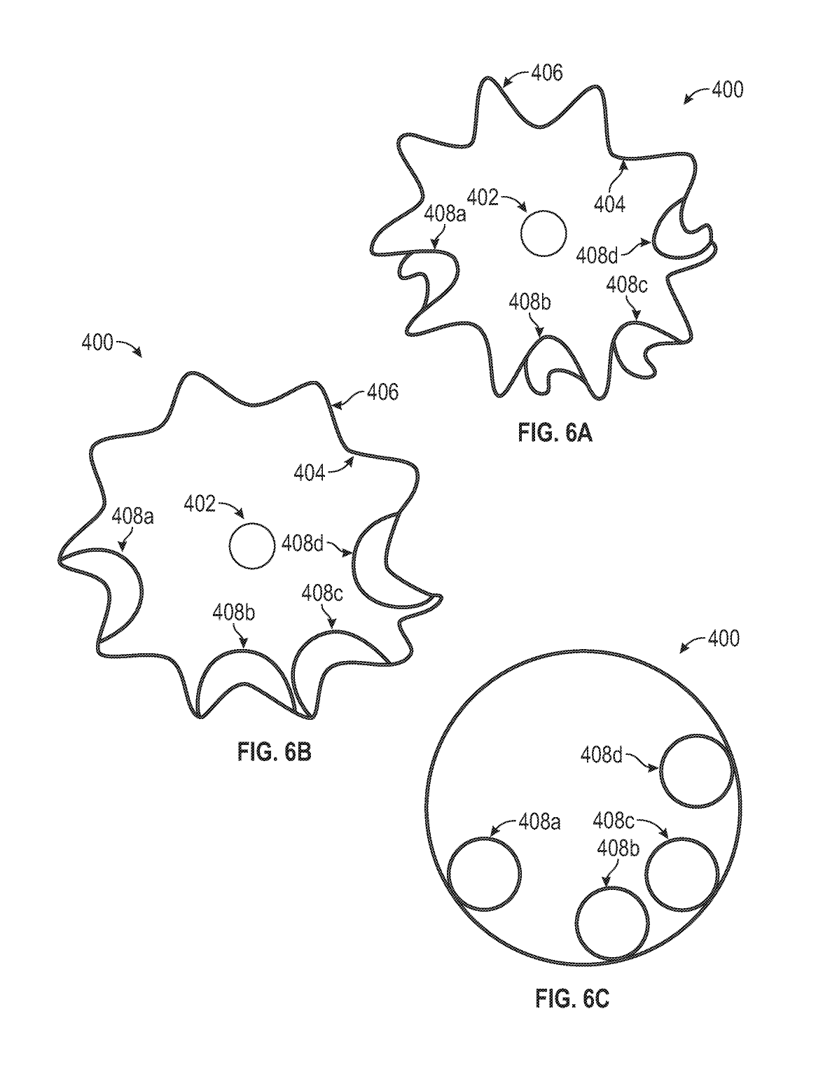

The disclosed endovascular graft designs, systems and methods are further illustrated and described with reference to the further schematic depictions described herein below. In particular, with reference to FIGS. 6A-6C, an exemplary endovascular graft 400 is shown in various stages of deployment. Specifically, FIG. 6A schematically depicts endovascular graft 400 in its initial, non-deployed configuration, whereas FIG. 6C schematically depicts endovascular graft 400 in a fully deployed configuration. FIG. 6B schematically depicts endovascular graft 400 at an intermediate point of deployment.

Endovascular graft 400 is positioned at a desired anatomical location by passing the graft along guidewire 402. Once positioned in a desired anatomical location, the stent structure 404 of graft 400 is outwardly expanded to such that the graft material 406 supported by the stent structure 404 engages the inner wall of a vessel/artery (not shown). Of note, four (4) distinct lumen- or chute-forming structures 408a, 408b, 408c, 408d are defined within the stent structure 404. Each of the lumen-/chute-forming structures 408a-408d is initially of reduced cross-section, thereby facilitating introduction of endovascular graft 400 to the desired anatomical location. However, as the endovascular graft 400 is deployed by expanding/inflating a balloon structure positioned therewithin, each of the lumen-/chute-forming structures 408a-408d expands/unrolls to ultimately define a substantially open (e.g., circular or oval) structure as schematically depicted in FIG. 6D. Once fully deployed, the guidewire 402 and inflation balloon (not shown) may be removed from the endovascular graft 400.

In FIGS. 6A-6D (and the succeeding schematic figures), the lumen-/chute-forming structures are schematically depicted as substantially cylindrical structures when fully expanded. However, it is to be understood that the lumen-/chute-forming structures may define an overlapping structure that is discontinuous, but defines in its expanded configuration a substantially cylindrical channel for introduction of stent-graft structure(s) and the like. Indeed, in exemplary embodiments of the present disclosure, the lumen-/chute-forming structure(s) are initially rolled within itself/themselves and assume an expanded position by unrolling. In the expanded position, the lumen-/chute-forming structures define passage(s) having a greater cross-sectional area as compared to the initial position in which it/they are rolled within itself/themselves.

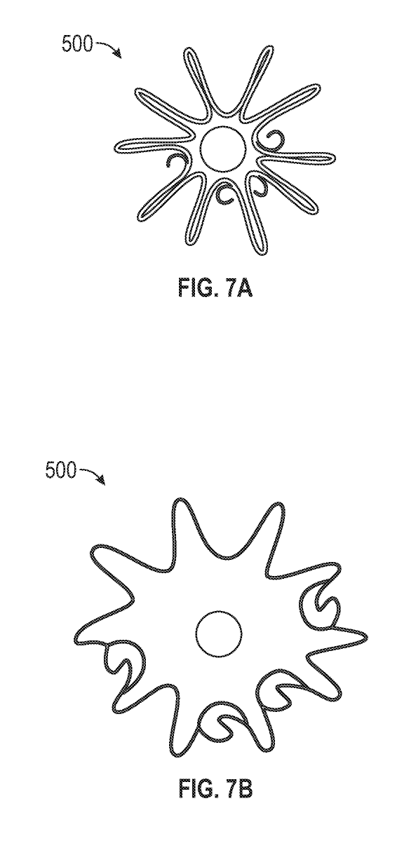

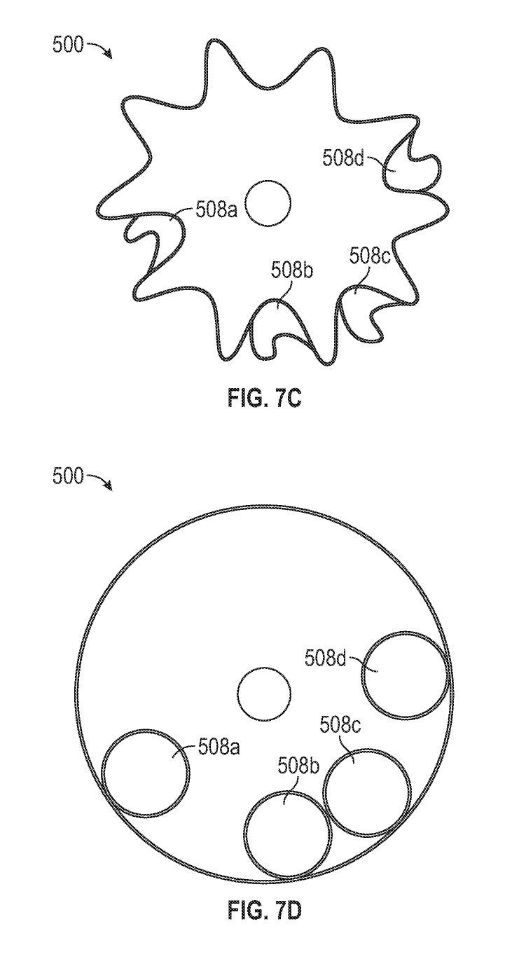

FIGS. 7A-7D provide a further series of schematic end views that illustrate the sequential deployment and expansion of an exemplary endovascular graft 500 according to the present disclosure. As is apparent, the overall cross-sectional size/dimension of the non-deployed endovascular graft 500 is significantly smaller (FIG. 7A) as compared to the fully deployed endovascular graft 500 (FIG. 7D). In addition, the lumen-/chute-forming structures 508a-508d initially occupy minimal space in terms of the cross-sectional size/dimension of endovascular graft 500 (FIG. 7A), but gradually expand (FIGS. 7B and 7C) until clinically effective lumens/chutes are defined within endovascular graft 500 (FIG. 7D).

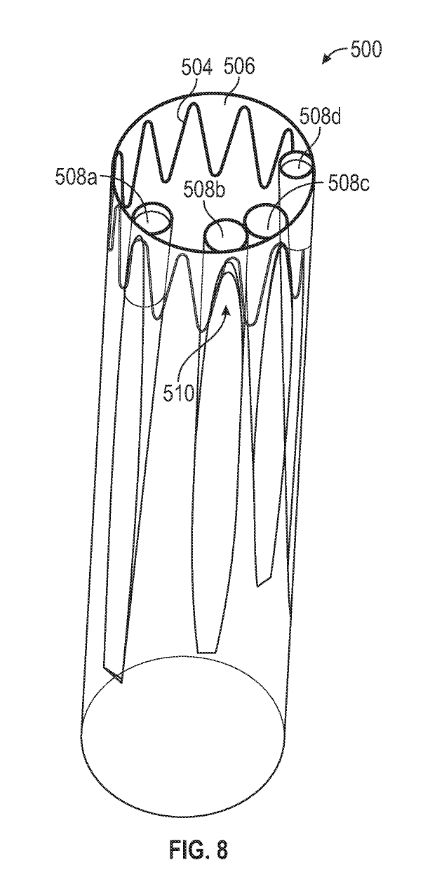

With reference to FIG. 8, exemplary endovascular graft 500 includes a stent structure 504 and an graft material 506 supported by the stent structure. A plurality of lumen-/chute-forming structures 508a, 508b, 508c, 508d are circumferentially spaced with respect to the endovascular graft 500 and each defines a fluted, outwardly opening passage 510 that permits passage of a stent graft to a branch vessel or artery. The fluted geometry of passage 510 is defined in part by the geometry of the stent structure 504, as shown in FIG. 8.

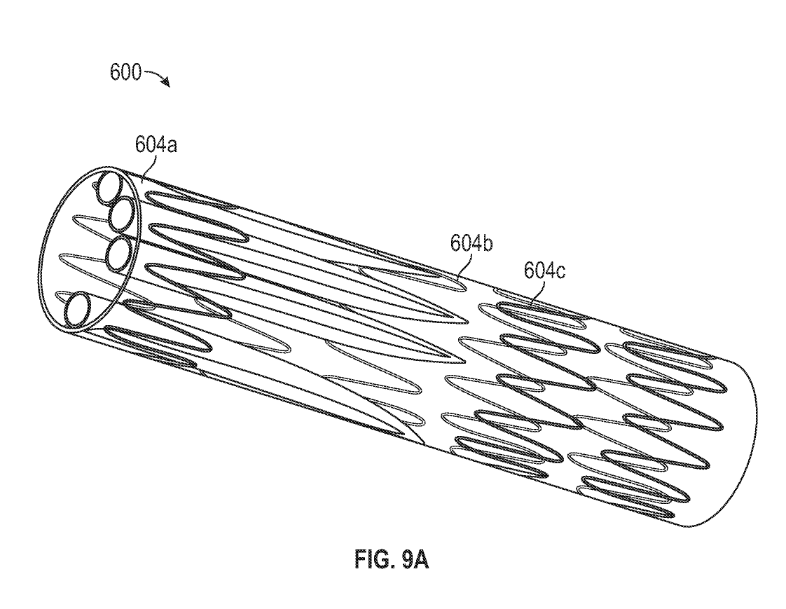

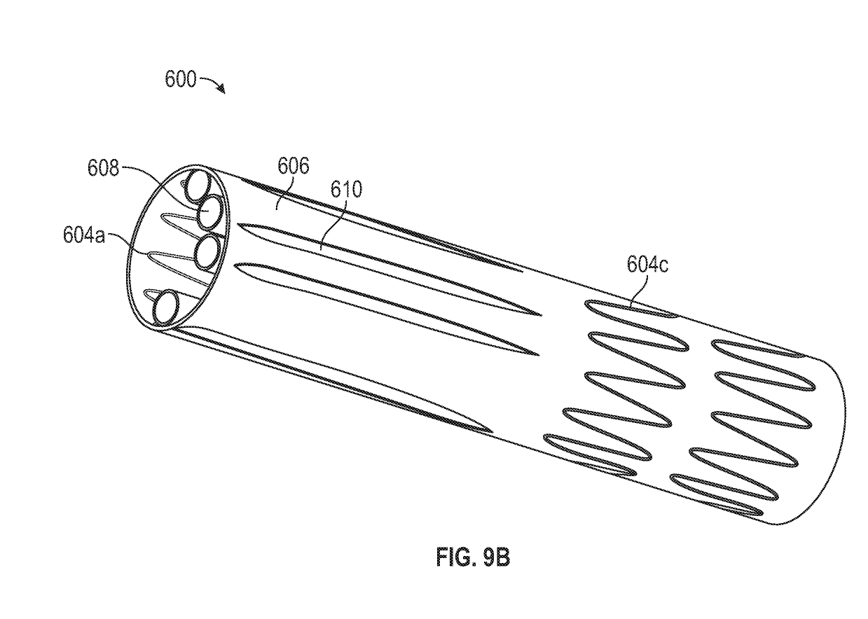

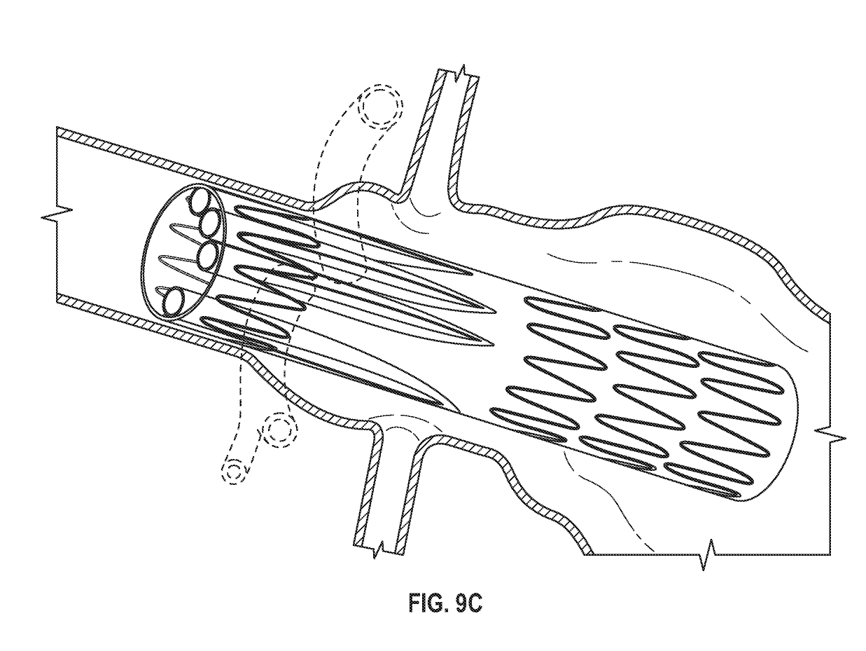

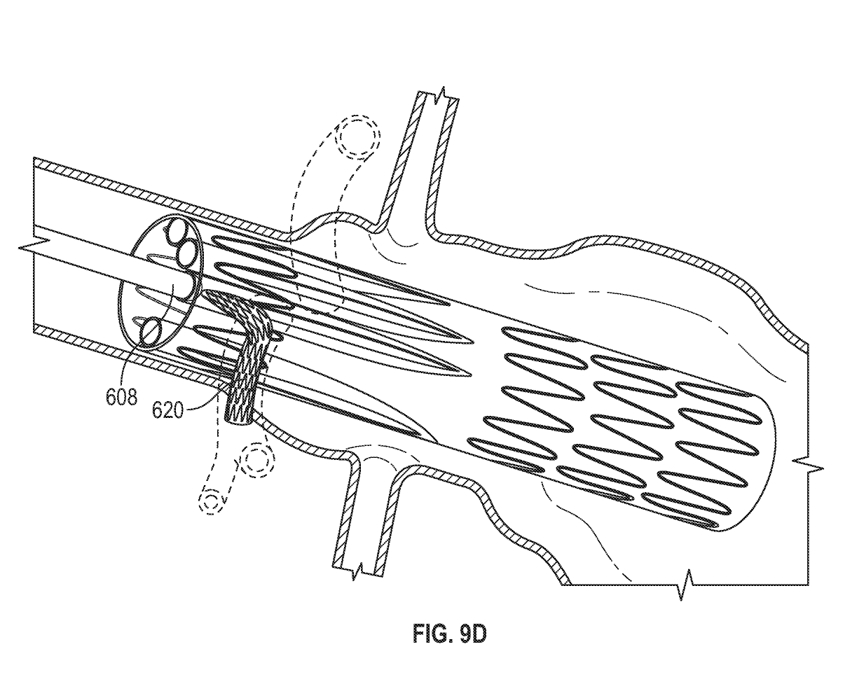

Turning to FIGS. 9A-9D, an exemplary endovascular graft 600 is schematically depicted in perspective views that illustrate various aspects of the present disclosure. In particular, FIG. 9A schematically depicts endovascular graft 600 with the outer graft material removed for illustration purposes. As shown in FIG. 9A, endovascular graft 600 may advantageously include a plurality of axially spaced stent structures 604a, 604b, 604c that cooperate to support the outer graft material. The number and spacing of the stent structures may vary from implementation-to-implementation and is generally determined by the desired axial dimension of the overall endovascular graft. The physical structure of the multiple stent structures are generally the same, although variations may be incorporated in the various stent structures, e.g., to provide desired dimensional parameters to the endovascular graft along its axial extent, as will be apparent to persons skilled in the art.

With reference to FIG. 9B, exemplary endovascular graft 600 is schematically depicted with the graft material 606 supported by stent structures 604a-604c. Fluted, outwardly opening passages 610 are defined by the lumen-/chute forming structures 606 to facilitate introduction of stent grafts to branch vessels/arteries.

FIG. 9C schematically depicts endovascular graft 600 with respect to anatomical structures and FIG. 9D shows introduction of an exemplary stent graft 620 to a branch vessel/artery through lumen-/chute-forming structure 608.

Although the present invention has been described with reference to exemplary embodiments and implementations thereof, it is to be understood that the disclosed endovascular graft designs, systems and methods may be modified, refined and/or enhanced without departing from the spirit or scope of the present disclosure. Further, any of the embodiments or aspects of the invention as described in the claims or in the specification may be used with one and another without limitation.

1. An endovascular graft, comprising:
a. a stent structure that is adapted to move between a collapsed and a deployed configuration,
b. an endovascular graft material mounted with respect to the stent structure,
c. at least one lumen- or chute-forming structure associated with the stent structure internal to the endovascular graft material, the at least one lumen- or chute-forming structure adapted to move between a first position and a second position, wherein the at least one lumen- or chute-forming structure is rolled within itself in the first position;
wherein the at least one lumen- or chute-forming structure moves from the first position to the second position by unrolling; and
wherein when in the second position, the at least one lumen- or chute-forming structure defines a passage having a greater cross-sectional area as compared to the first position.

2. The endovascular graft of claim 1, wherein the stent structure defines a longitudinal axis and wherein the at least one lumen- or chute-forming structure is physically joined to the stent structure along the longitudinal axis.

3. The endovascular graft of claim 2, wherein the stent structure and the at least one lumen- or chute-forming structure are integrally formed by a laser cutting operation.

4. The endovascular graft of claim 1, wherein the at least one lumen- or chute-forming structure includes a plurality of lumen- or chute-forming structures.

5. The endovascular graft of claim 4, wherein the plurality of lumen- or chute-forming structures are radially spaced relative to the stent structure.

6. The endovascular graft of claim 5, wherein the radial spacing of the plurality of lumen- or chute-forming structures is radially equidistant.

7. The endovascular graft of claim 5, wherein the radial spacing of the plurality of lumen- or chute-forming structures is not radially equidistant.

8. The endovascular graft of claim 4, wherein when in the second position, the plurality of lumen- or chute-forming structures define passages with substantially equal cross-sectional areas.

9. The endovascular graft of claim 4, wherein when in the second position, the plurality of lumen- or chute-forming structures define passages with substantially unequal cross-sectional areas.

10. The endovascular graft of claim 1, wherein the at least one lumen- or chute-forming structure is biased to move from the first position to or toward the second position.

11. The endovascular graft of claim 1, wherein in the second position, the at least one lumen- or chute-forming structure defines a cross-section that is substantially circular.

12. The endovascular graft of claim 1, wherein when in the second position, the at least one lumen- or chute-forming structures defines a passage that is substantially cylindrical in geometry.

13. The endovascular graft of claim 1, wherein the lumen- or chute-forming structure defines a substantially interrupted cylindrical geometry.

14. A method for treating vasculature, comprising:
a. providing an endovascular graft in a collapsed/non-deployed configuration;
b. positioning the endovascular graft within a main artery; and
c. deploying the endovascular graft within the main artery, such that the endovascular graft assumes an expanded/deployed configuration and wherein one or more lumens/passages are defined within the endovascular graft to allow stent-graft deployment to one or more branch arteries/vessels;
wherein the one or more lumens/passages are an initial configuration rolled within itself or themselves; and
wherein the one or more lumens/passages unroll from the initial rolled configuration to an unrolled configuration as the endovascular graft is deployed within the main artery to assume the expanded/deployed configuration.

15. The method of claim 14, further comprising deployment of one or more stent-grafts in one or more branch arteries/vessels through the one or more lumens/passages defined within the endovascular graft.

16. The method of claim 14, wherein the endovascular graft defines a plurality of lumens/passages therewithin.

17. The method of claim 14, wherein the endovascular graft engages an inner wall of a vessel when in the expanded/deployed configuration, and wherein blood leakage around the expanded/deployed configuration is substantially prevented.
